(19) United States
(12) Patent Application Publication
(10) Pub. No.: US 2006/0004392 A1
(43) Pub. Date: Jan. 5, 2006

(54) Anastomosis clamp

(76) Inventor: Paul D. Amarant, Davie, FL (US)

Correspondence Address: BRINKS HOFER GILSON & LIONE/INDY/COOK, ONE INDIANA SQUARE, SUITE 1600, INDIANAPOLIS, IN, 46204-2033, US

(21) Appl. No.: 11/121,604

(22) Filed: May 4, 2005

Related U.S. Application Data
(60) Provisional application No. 60/570,661, filed on May 13, 2004.

(51) Int. Cl. A61B 17/08

(52) U.S. Cl. 606/153

(57) ABSTRACT

A clamp for use in joining two vessels in end-to-end anastomosis. The clamp comprises a clamp body comprising opposing clamp members joined by a hinge. The clamp members are pivotable along the hinge to selectively maneuver the clamp between an open position and a closed position. Each of the opposing clamp members includes an opening sized for axial passage of at least a portion of a separate one of the vessels therethrough. The clamp body includes a closing mechanism for sealing the clamp when the clamp members are pivoted to the closed position, whereupon the vessels are aligned to establish fluid communication therebetween.

### RELATED APPLICATION

The present patent document claims the benefit of the filing date under 35 U.S.C. §119(e) of Provisional U.S. Patent Application Ser. No. 60/570,661, filed May 13, 2004, which is hereby incorporated by reference.

### BACKGROUND

1. Technical Field

The present invention relates to a clamp for joining attachable tissue or graft segments, and more particularly, to a clamp for end-to-end anastomosis of medical grafts, blood vessels, and the like.

2. Background Information

Anastomosis is the joinder of hollow vessels to create an internal communication between them. An anastomosis is generally created by a surgical procedure that joins two body vessels, vascular grafts, or a body vessel and a graft, in order to create or restore a pathway for fluid flow through the joined structure. Commonly, an anastomosis is created by vascular surgery to join two blood vessels, grafts, or a blood vessel and a graft, to create or restore blood flow therethrough.

Current devices and techniques exist which allow for open-ended surgical attachment of harvested body vessels or grafts for purposes such as the avoidance of a vessel blockage, replacement of diseased vessels, and vascular access. Such techniques include sewing or otherwise attaching a vessel or graft between open ends of existing vessels. Examples of vessel pairs which are frequently joined by a vessel or graft include an internal mammary artery and a coronary vessel, the radial artery and cephalic vein, the brachial artery and cephalic vein, the brachial artery and basilica vein, the ulnar artery and a basilica vein, and a brachial artery and branches of the antecubital vein, among others.

It is generally preferred to join such vessels utilizing the patient's natural vessels. This connection may be between two natural vessels positioned in their natural place of orientation in the body, or alternatively, utilizing one or more natural vessels harvested from another portion of the patient's anatomy. Utilizing a vessel harvested from another portion of the patient's anatomy minimizes the possibility that the patient will experience incompatibility or rejection problems of the type that may occur when using graft materials that originate from an external source, or from using exogenous tissue. In addition, such harvested vessels provide a ready supply of biological tissue that has already proven to be biologically compatible with the patient.

At times, however, suitable body vessels may not be available for harvesting. In such cases, a synthetic vessel (e.g., TEFLON® or DACRON®) or an exogenous vessel may be used. Synthetic vessels have been found to be effective in many instances. However, such vessels have shown a greater propensity to become narrowed than do natural arteries or veins. Exogenous vessels may also be utilized in an appropriate case. However, there is a greater likelihood of patient incompatibility with such vessels when compared to natural vessels harvested from the patient.

Many different types of anastomosis connections between a vessel and a graft are known in the medical arts. For example, an anastomosis connection may be utilized to join vessels from the end of a graft to the side of a vessel, commonly referred to as an end-to-side connection. An anastomosis connection may also be utilized to join the end of a graft to the end of a vessel, commonly referred to as an end-to-end connection. A side-to-side connection of a vessel and a graft may also be established. This type of connection is commonly referred to as a fistula.

End-to-end connections are generally considered beneficial because they essentially mimic the normal flow of fluid through the natural vessel. With regard to such end-to-end connections, however, it is important to insure that a secure and leak-free connection be established.

Prior art connection devices are at times unsecure, and also have been prone to leakage. While synthetic joinder materials are available, such materials are often complicated and difficult to use. A need exists for an improved device for end-to-end connection that provides a secure and leak-free connection, that is relatively easy for the surgeon to manipulate and insert, and that is cost-effective.

### BRIEF SUMMARY

The present invention addresses the problems of the prior art by providing an anastomosis clamp for securely clamping together segments of medical grafts, body vessels and/or tubular conduits.

In one embodiment, the present invention comprises a clamp for use in joining vessels in end-to-end anastomosis. The clamp body includes opposing clamp members joined by a hinge such that the clamp members are pivotable along the hinge between an open position and a closed position. Each of the opposing clamp members includes an opening sized for axial passage of at least a portion of one of the vessels therethrough. The clamp body includes a closing mechanism for sealing the clamp when the clamp members are pivoted to the closed position.

In another embodiment, the present invention comprises a method for joining vessels in end-to-end anastomosis. A clamp body has opposing clamp members joined by a hinge, such that the clamp members are pivotable between an open position and a closed position. Each of the opposing clamp members includes an opening sized for axial passage of at least a portion of one of the vessels. The clamp body further includes a closing mechanism for closing the clamp when the clamp members are pivoted to the closed position. First and second vessels are provided for joinder, each of the vessels preferably including a flanged portion at an axial end of the vessel. The first vessel is aligned with the clamp body such that its flanged portion abuts an inner surface of one of the clamp members, and the reminder of the vessel extends axially through the opening of the clamp member. The second vessel is aligned with the clamp body such that its flanged portion abuts an inner surface of the other clamp member, and the reminder of the vessel extends axially through the opening of the other clamp member. The hinge is then pivoted such that the clamp members are in the closed position and fluid communication is established between the vessels. The clamp members may be locked in the closed position to maintain fluid communication between the vessels.

### BRIEF DESCRIPTION OF THE DRAWINGS

FIG. 1 is a front view of a clamp according to an embodiment of the present invention, shown in an open position;

FIG. 2 is side view of a vessel of the type to be clamped together with another vessel to establish an anastomosis;

FIG. 3 is a side view of the vessel of FIG. 2, provided with a flange at an axial end thereof;

FIG. 4 is a perspective view of the inventive clamp in the closed position joining two vessels; and

FIG. 5 is a side view of the inventive clamp, in the closed position clamping two vessels.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

The present invention comprises a percutaneous anastomosis clamping apparatus for establishing an end-to-end anastomosis connection between two hollow structures in the body. The particular connections resulting from use of the inventive apparatus may be, for example, a graft-to-graft connection, a vessel-to-graft connection, or a vessel-to-vessel connection. For a vessel-to-vessel connection, the connection may be established between natural vessels, exogenous vessels, synthetic vessels, or any combination of the foregoing.

Although it is expected that the clamping apparatus will normally be utilized to connect blood vessels, other body vessels may also be joined to vessels, other body structures, grafts, synthetic or exogenous vessels. One non-limiting example of a connection of this type comprises the connection of the ureter vessel to the urethra. For applications in bodily systems, such as the circulatory system, ultrasound guidance can be utilized to help establish connection between the apparatus and other structures in the system, such as an artery, a vein, or both an artery and a vein. Those skilled in the art will appreciate that other bodily connections can be made using the clamping apparatus and method of the present invention, and that medical guidance systems other than ultrasound may be utilized in an appropriate case, all of which are considered within the scope of the invention.

Such hollow body vessels, such as blood vessels, are joined in a manner to permit or restore fluid flow therebetween. The anastomosis connection provides a means to bridge the vessels within the body of a patient in end-to-end fashion. The term “vessel” is used herein in inclusive fashion to include body vessels or other hollow structures (both endogenous and exogenous), medical grafts, synthetics, and other segments that may be joined by the apparatus of the present invention.

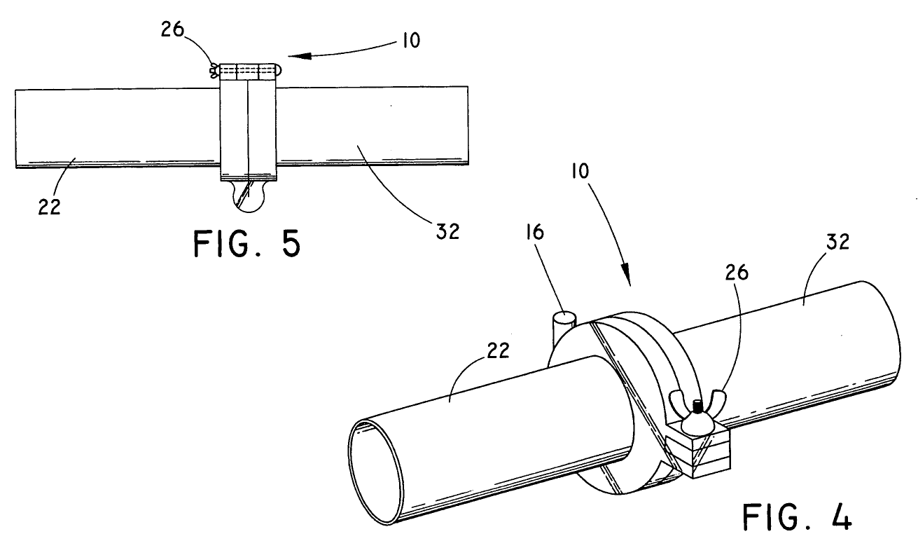

One embodiment of an anastomosis clamping apparatus for joining two vessels in an end-to-end anastomosis connection is shown in FIG. 1. In this figure, clamp 10 is shown in its “open” position. Clamp 10 may be formed of any conventional medical grade components, such as a plastic, metal, metal alloy, composite construction, or a combination thereof, that is suitable for implantation in a human or other animal. Clamp 10 includes two clamp portions 12, 14 joined by a conventional pivotable hinge 16. Pivotable hinge 16 enables clamp 10 to be selectively pivoted between the open position shown in FIG. 1 and a closed position. FIGS. 4 and 5 illustrate the clamp in the closed position. The type of hinge 16 utilized as a part of clamp 10 is not restricted, and any conventional type of hinge may be utilized. Preferably, hinge 16 comprises a living hinge or a riveted hinge.

Clamp 10 is provided with a conventional closing mechanism 26 opposite hinge 16 to establish closure of the clamp. The closing mechanism 26 can comprise any well-known mechanism used to establish closure of a hinge, and that is capable of forming a reliable seal. Non-limiting examples of such closing mechanisms include a screw and wingnut combination, a press fit, a set screw, or an adhesive. The screw and wingnut combination is shown in the drawings.

Clamp 10 may also be provided with an optional channel, such as beveled channel 18 shown in FIG. 1, that is positioned along the inner surface of either or both of clamp portions 12, 14. An O-ring 20 or similar compressive article may be positioned within beveled channel 18. When the clamp is in its closed position, compression of the 0-ring enhances the seal formed by the clamp in well-known fashion.

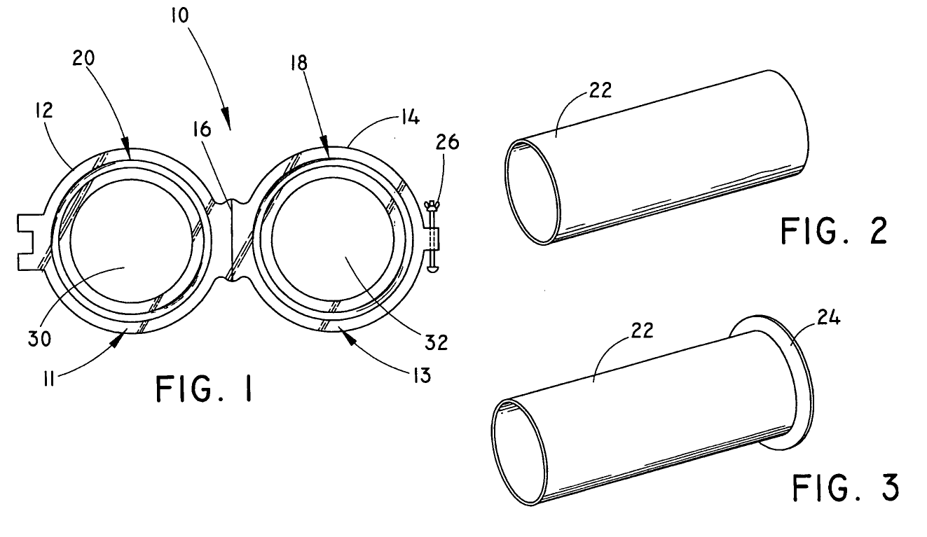

FIG. 2 illustrates a vessel 22 of the type that can be joined to another vessel or like structure by end-to-end anastomosis utilizing the inventive clamp. Although referred to herein for convenience as a vessel, those skilled in the art will appreciate that any of the types of segments that are conventionally joined by anastomosis techniques, such as a body vessel, medical graft, and other endogenous or exogenous tissue segment, are included in the description.

Prior to placing vessel 22 into clamp 10, it is preferred to incorporate a flange 24 onto at least one of the vessels. A vessel 22 having a flange 24 attached is shown in FIG. 3. Flange 24 may be formed by manipulating an axial end of vessel 22 to extend radially from the vessel. Alternatively, an external flange formed of a biologically compatible composition suitable for implantation can be incorporated onto the axial end of the vessel by conventional attachment techniques. The presence of flange 24 assists in anchoring the vessel in the clamp and minimizes the possibility of dislodgement of the vessel while in the clamp.

Use of the inventive clamp to join vessels 22, 32 will now be described. Clamp 10 is initially manipulated to the open position shown in FIG. 1. Vessel 22 is aligned such that flanged end 24 abuts the inner surface 11 of clamp 10, and the remaining body of vessel 22 projects outwardly in an axial direction through clamp opening 30. This may be best observed in the orientation of FIGS. 4 and 5. Likewise, vessel 32 is aligned such that its flanged end (not shown) abuts the inner surface 13 of clamp 10, and the remaining body of vessel 32 projects outwardly in an axial direction through clamp opening 32, opposite the direction of vessel 22.

Hinge 16 is then pivoted to close clamp 10. In the closed position (FIGS. 4 and 5), the respective flanged ends of vessels 22, 32 will preferably be in abutment with each other. The closing mechanism 26 is activated to seal the clamp in the closed position. The anastomosis clamp thereby provides an end-to-end joinder of vessels 22, 32. It most cases it will be permissible if a radial portion of the flange of vessel 22 and/or 32 extends beyond the diameter of the vessel hanging outside in the open space. Thus, it is generally not necessary to flange the vessel to fit precisely inside the clamp.

Although the inventive apparatus has been described for use in joining two blood vessels, those skilled in the art will recognize that other known components can likewise be joined, such as other vessels, other body structures wherein a pathway is to be established (or re-established) therebetween, synthetic graft materials, exogenous materials, ands combinations of them. Likewise, a blood vessel may be attached to a synthetic graft vessel or an exogenous vessel. In addition, the invention is not limited to vascular access, but rather, may also include other applications. Non-limiting examples of such applications include bypass grafting between two blood vessels, including fem-fem (femoral artery and femoral vein) and fem-pop; coronary artery bypass grafting; and shunting outside of the circulatory system to help alter flow of fluid including,gastrointestinal tract (e.g., liver and gall bladder), the urinary system (e.g., ureter and urethra), beyond the blood-brain barrier (e.g., for hydroencephalopathy), and in the reproductive system (e.g., ovarian recannulation).

It is therefore intended that the foregoing detailed description be regarded as illustrative rather than limiting, and that it be understood that it is the following claims, including all equivalents, that are intended to define the spirit and scope of this invention.

1. A clamp for use in joining two vessels in end-to-end anastomosis, comprising:
a clamp body comprising opposing clamp members, said opposing clamp members being joined by a hinge such that said clamp members are pivotable along said hinge to selectively maneuver said clamp between an open position and a closed position, each of said opposing clamp members including an opening sized for axial passage of at least a portion of one of said vessels therethrough, said clamp body including a closing mechanism for sealing said clamp when said clamp members are pivoted to said closed position, whereby said vessels are aligned when the clamp is in the closed position such that fluid communication may be established therebetween.

2. The clamp of claim 1, wherein said hinge comprises a living hinge.

3. The clamp of claim 1, wherein said closing mechanism comprises a screw and wingnut.

4. The clamp of claim 1, wherein each one of said clamp members has a surface sized and shaped for registry with a surface of the other of said clamp members when said clamp is in the closed position.

5. The clamp of claim 4, wherein the surface of at least one of said clamps includes a channel.

6. The clamp of claim 5, wherein the surface of each of said clamps includes a channel, said channels positioned such that the channels are aligned to comprise a groove when said clamp is in the closed positioned.

7. The clamp of claim 6, wherein said clamp further comprises an O-ring receivable in said groove.

8. The clamp of claim 1, wherein at least one of said vessels aligned for joinder in said clamp includes a flange at an end thereof, said flange having a greater radial diameter than a diameter of said opening.

9. The clamp of claim 8, wherein each of said vessels aligned for joinder in said clamp includes a flange at an end thereof, said flanges having a greater radial diameter than a diameter of said opening.

10. A method for joining two vessels in end-to-end anastomosis to provide a fluid pathway therebetween, comprising:
providing a clamp body comprising opposing clamp members, said opposing clamp members being joined by a hinge such that said clamp members are pivotable along said hinge between an open position and a closed position, each of said opposing clamp members including an opening sized for axial passage of at least a portion of one of vessels therethrough;
aligning a first one of said vessels with one of said clamp members such that a portion of said first vessel extends axially through the opening of said clamp member;
aligning a second one of said vessels with the other of said clamp members such that a portion of said second vessel extends axially through the opening of said other clamp member; and
pivoting said hinge to said closed position, said openings of said opposing clamp members being substantially aligned when said hinge is in said closed position such that a passageway in said first vessel is in fluid communication with a passageway in said second vessel.

11. The method of claim 10, wherein said clamp body includes a mechanism for sealing said clamp when said clamp members are pivoted to said closed position, said method further comprising the step of activating said mechanism for sealing said clamp body in said closed position to maintain said fluid pathway between the vessels.

12. The method of claim 11, wherein each of said clamp members has a first surface and a second surface, said first surface of each respective clamp member being in facing relationship when said clamp body is in said closed position, wherein each of said vessels aligned for joinder in said clamp includes a flange at an end thereof, said flanges being disposed between said respective first faces when said clamp body is in said closed position.

13. The method of claim 11, wherein said hinge comprises a living hinge.

14. The method of claim 11, wherein said closing mechanism comprises a screw and wingnut.

15. The method of claim 11, wherein each one of said clamp members has a surface sized and shaped for registry with a surface of the other of said clamp members when said clamp is in the closed position.

16. The method of claim 15, wherein the surface of at least one of said clamps includes a channel.

17. The method of claim 16, wherein the surface of each of said clamps includes a channel, said channels positioned such that the channels are aligned to comprise a groove when said clamp is in the closed positioned.

18. The method of claim 17, wherein said clamp further comprises a compressible article in said groove.

19. The method of claim 18, wherein said compressible article comprises an O-ring receivable in said groove.

20. The method of claim 10, wherein each of said vessels aligned for joinder in said clamp includes a flange at an end thereof, said flanges having a greater radial diameter than a diameter of said openings.
